(19) United States
(12) Patent Application Publication
(10) Pub. No.: US 2026/0017448 A1
(43) Pub. Date: Jan. 15, 2026

(54) SYSTEM AND METHOD FOR CONVERTING AUDIO DATA INTO AN ANNOTATED SUMMARIZATION

(71) Applicant: MH SUB I, LLC, El Segundo, CA (US)

(72) Inventor: Amit Prasad, Los Angeles, CA (US)

(21) Appl. No.: 19/261,714

(22) Filed: Jul. 7, 2025

Related U.S. Application Data
(60) Provisional application No. 63/670,041, filed on Jul. 11, 2024.
(60) Provisional application No. 63/711,619, filed on Oct. 24, 2024.

(51) Int. Cl. G06F 40/169; G06F 40/109; G06F 40/279; G16H 10/60

(52) U.S. Cl. CPC G06F 40/169; G06F 40/109; G06F 40/279; G16H 10/60

(57) ABSTRACT

A computing device operating with generative artificial intelligence (AI) logic to generate a natural language processing (NLP) prompt from a text-based transcript converted from audio data captured by the computing device, receive a summary from the generative AI logic, recognize one or more keywords within the summary, and annotate the one or more keywords by at least modifying an appearance of the one or more keywords or appending information to give the one or more keywords prominence during a visual review of the summary.

### CROSS-REFERENCE TO RELATED APPLICATIONS

This application claims the benefit of priority on both U.S. Provisional Applications No. 63/670,041 filed Jul. 11, 2024 and U.S. Provisional Application No. 63/711,619 filed Oct. 24, 2024, the entire contents of both of these applications are incorporated by reference herein.

### FIELD

Embodiments of the disclosure relate to the field of artificial intelligence (AI) platform utilization. More specifically, one aspect of the disclosure relates to a system and method that applies annotations on content that has been translated from audio data into text data and summarized by generative AI logic.

### GENERAL BACKGROUND

Generative AI technology has been recently deployed as an intelligent agent to conduct conversations with human users. For example, large language models (LLMs) such as ChatGPT for example, have provided a conversational artificial intelligence (AI) platform to perform natural language processing (NLP) tasks. Recently, organizations are beginning to submit documents directly to LLMs and instructing them to generate an output that describes content of the document in a more concise format (hereinafter, a “summary”). However, summaries currently produced by LLMs have experienced quality issues, especially when the original content is based on text content converted from audio content.

For instance, a recorded consultation between a patient and a medical professional can be processed through a voice-to-text recorder to generate a transcript of the consultation. However, it is foreseeable that an audio-to-text converter may incorrectly identify a spoken medicinal dosage of “fifteen milligrams” as “fifty milligrams,” especially when the speaker's word enunciation is poor or the speaker is facing away from the microphone of the computing device recording the dialogue between the patient and her physician. This incorrect dosage would be reflected in the transcript, and any summaries of that transcript generated by an LLM given prompted by such a transcript would likely replicate the incorrect medicinal dosage. If the physician or other medical personnel fails to detect the erroneous dosage value within the summary produced by generative AI logic, this oversight could lead to significant safety issues for patients and liability concerns for physicians.

A mechanism is needed to better assist physicians and/or other healthcare professionals in reviewing summaries of physician-patient meetings concerning medical treatment, most notably number/dosage/usage metrics associated with a prescribed medicinal treatment for the patient. This annotation of the summary is designed to assist physicians by improving the accuracy of electronic health records and also greatly reduce the burden of checking every detail and give medical personnels valuable time back to deal with other patients.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an exemplary block diagram of a computing device with summary coordination logic configured to capture audio, convert the audio into text content, and perform optional anonymization of the text content prior to delivery to a cloud service for summary generation and subsequent annotation of the resulting summary according to some examples.

FIG. 2 is an exemplary flowchart illustrating the operability of the AI summarization workflow tool of FIG. 1 according to some examples.

FIG. 3 is an exemplary flowchart further illustrating an embodiment of the summary generation and annotation process implemented by the summary coordination logic of FIG. 1 according to some examples.

FIG. 4 is an exemplary flowchart illustrating an embodiment of the anonymity control process implemented by the summary coordination logic of FIG. 1 according to some examples.

FIG. 5 is an exemplary flowchart illustrating an embodiment of keyword flagging and annotation process implemented by the summary coordination logic of FIG. 1 according to some examples.

FIG. 6A is an exemplary perspective view of a computing device adapted with the summary coordination logic of FIG. 1.

FIG. 6B is an exemplary embodiment of the computing device after the recorded session has completed in which a graphic user interface (GUI) generation logic is configured to produce a visualization that allows the user to select a summary template.

FIG. 6C is an exemplary embodiment of the visualization of FIG. 6B in which the user may select a summary template that is a predetermined template structure or a summary template that is customized by the user.

FIG. 6D is an exemplary embodiment of one of the summary templates populated with the transcript data within different sections that may be modifiable by the user on the computing device, or another computing device communicatively coupled to that computing device.

### DETAILED DESCRIPTION

Various embodiments of the disclosure are directed to summary (content) coordination logic configured with transcript generation logic, prompt generation logic, and/or summary (content) modification logic. Herein, the transcript generation logic is configured to convert captured audio data into a transcript (e.g., text content associated with the audio session). The prompt generation logic is designed to create a natural language processing (NLP) prompt that is transmitted to a content summarization service, where the prompt includes the transcript. Lastly, the summary (content) modification logic is configured to parse a summary received from the content summarization service, which includes generative AI logic, such as large language models (LLMs) as described below, to identify and annotate salient content within the summary prior to storage and/or display for review by the user. If anonymization is conducted, the summary (content) modification logic may identify the anonymized data and return such data into its original form prior to parsing the summary.

According to another embodiment of the disclosure, the summary coordination logic may further include an anonymity control logic, which is configured to perform anonymization of the transcript to remove sensitive content directed to one or more of the participants of the recorded meeting such as individually identifiable health information from a doctor-patient consultation (e.g., patient name, birth date, address, and social security number). The anonymization is conducted prior to delivery of the transcript to a content summarization service. The operability of the anonymity control logic is described herein for clarity, although it is contemplated that such functionality does not need to be deployed.

Certain functionality enabled by the summary coordination logic may include, but is not limited or restricted to the following:
1. Automatic Transcription: Embodiments of the invention may be configured to facilitate the recording of doctor-patient consultations, converting spoken words into text format efficiently and accurately.
2. Medical Terminology Understanding: The summary coordination logic possesses the capability to comprehend and interpret medical jargon and terminology in order to enhance its transcription accuracy and usefulness in medical settings. The summary coordination logic is further adapted to analyze and annotate (e.g., highlight) key items physicians or other health care professionals should double check after their patient visit. Any recording/transcription operation has the ability to misinterpret certain terminology, including terminology that may pose safety concerns such as recommended medical treatments and their frequency or prescription information such as drug name, dosage, and/or intake frequency that were discussed during an actual appointment. Summary coordination logic is configured to bring prominence to such content to assist the physicians or other health care professionals in the summary review process.
3. Summary Generation: The summary coordination logic is a software-based tool configured to assist in generating concise summaries of consultations, condensing lengthy discussions into key points for easier review and reference.
4. Auto-Linking to Medical Information: The summary coordination logic may be configured to automatically generate and insert links to relevant medical conditions and guidelines, providing physicians or other health care professionals with quick access to additional information, thereby enhancing decision-making and saving time.

### I. Terminology

In the following description, certain terminology is used to describe aspects of the invention. For example, in certain situations, the terms “logic,” “element,” and “component” are representative of hardware, firmware, or software that is configured to perform one or more functions. As hardware, logic (or element or component) may include circuitry having data processing or storage functionality. Examples of such circuitry may include, but are not limited or restricted to, one or more hardware processors (e.g., a microprocessor with one or more processor cores, a digital signal processor, a programmable gate array, a microcontroller, an application specific integrated circuit “ASIC,” etc.), a semiconductor memory, or combinatorial elements.

Alternatively, logic (or element or component) may be software, such as executable code in the form of an executable application, a graphical user interface (GUI), an Application Programming Interface (API), a subroutine, a function, a procedure, an applet, a servlet, a routine, source code, object code, a shared library/dynamic library, or one or more instructions. The software may be stored in any type of a suitable non-transitory storage medium or transitory storage medium (e.g., electrical, optical, acoustical, or other forms of propagated signals such as carrier waves, infrared signals, or digital signals). Examples of the non-transitory storage medium may include, but are not limited or restricted to, a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device.

A “computing device” may be generally construed as electronics with data processing capability and/or a capability of connecting to any type of network, such as a public network (e.g., Internet), a private network (e.g., a wireless data telecommunication network, a local area network “LAN,” etc.), or a combination of networks. Examples of a computing device may include, but are not limited or restricted to, the following: a server, an endpoint device (e.g., a laptop, a smartphone, a tablet, a desktop computer, a netbook, networked wearable, or any general-purpose or special-purpose, user-controlled electronic device); a mainframe; a router; or the like.

The terms “annotate,” “annotation,” or other tenses thereof identify a modification of content to attract attention by a user to such content. For example, an annotation may constitute (a) highlighting, (b) a change in font style (e.g., italicize, bold, underline, etc.), (c) a change in font type or size (e.g., Times New Roman, Arial, Calibri, etc.), (d) a change in font color (e.g., change from normal black to red, green, blue, etc.), (e) attachment of a comment to the annotated term that can be deleted after review, or the like.

A “transcript” may be generally construed as a collection of text content converted from audio, which may be processed into a summary. A “summary” refers to a condensed version of the transcript (i.e., lesser number of characters or storage size as bytes, kilobytes, or megabytes, etc.) that summarizes the transcript. The term “salient,” when referenced in connection with the summary, identifies the importance of the content-signifying the accuracy of such data is importance to the accuracy of the electronic health record and safety of the patient.

A “message” generally refers to information transmitted in one or more electrical signals that collectively represent electrically stored data in a prescribed format. Each message may be in the form of one or more packets, frames, HTTP-based transmissions, or any other series of bits having the prescribed format. The message may include a “prompt,” namely a piece of text or code that serves as input for generative AI logic such as a large language model (LLM) for example. The prompt can be used to generate various types of content, such as text, images, or even code that form a portion of the summary.

The term “computerized” generally represents that any corresponding operations are conducted by hardware in combination with software and/or firmware.

Lastly, the terms “or” and “and/or” as used herein are to be interpreted as inclusive or meaning any one or any combination. Therefore, “A, B, or C” or “A, B, and/or C” mean “any of the following: A; B; C; A and B; A and C; B and C; A, B, and C.” An exception to this definition will occur only when a combination of elements, functions, steps, or acts are in some way inherently mutually exclusive.

### II. Artificial Intelligence Based (AI-Based) Summarization Platform

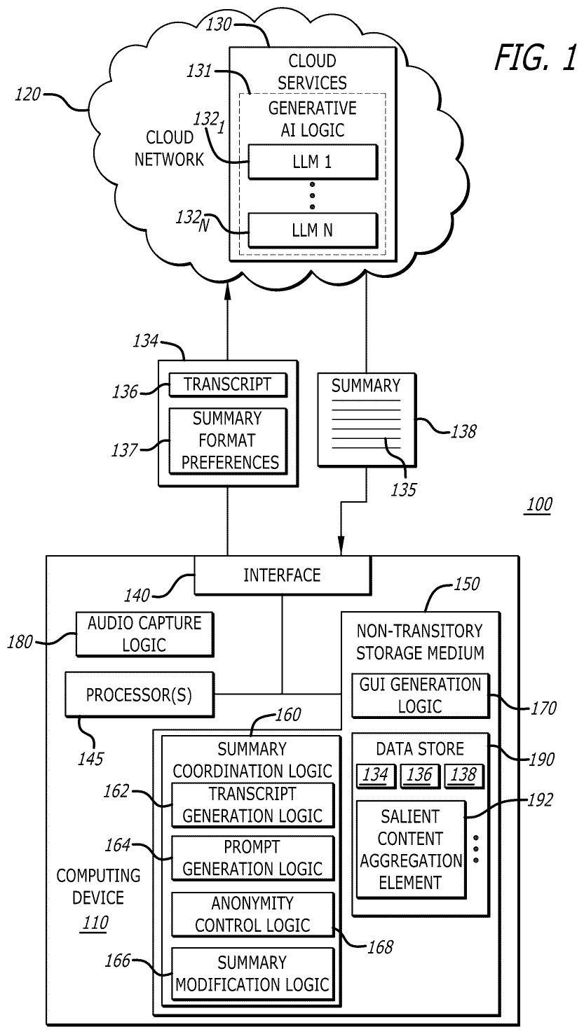

Referring to FIG. 1, an exemplary embodiment of an artificial intelligence based (AI-based) summarization platform 100 is shown. The AI-based summarization platform 100 includes a computing device 110 configured to communicate with a content summarization service 130 implemented as on-premises hosted service or as a cloud service deployed within a cloud network 120 such as a public cloud network or a private cloud network for example. Stated differently, the content summarization service 130 may be hosted locally within an application running on a computing device or may be implemented as a cloud-based service accessed through an application programming interface (API). According to one embodiment of the disclosure, the content summarization service 130 may include generative AI logic 132, such as one or more large language models (LLMs) 132₁-132_{N} (N≥1) for example.

As further shown in FIG. 1, the generative AI logic 132 may be adapted to receive a prompt 134 from the computing device 110, where the prompt 134 includes a transcript 136, namely an audio-to-text conversion. The prompt 134 may further include summary format preferences 137, which may be utilized by the generative AI logic 132 in producing a summary 138 of the transcript 136 in accordance with a format represented by the summary format preferences 137.

According to one embodiment of the disclosure, the computing device 110 features an interface 140, one or more processors 145 (hereinafter, “processor(s)”), and a non-transitory storage medium 150. The interface 140 is adapted to support communications with the content summarization service 130, which may be deployed as a service within the cloud network 120 as shown. The processor(s) 145 is adapted to execute software that at least partially controls the operability of the AI-based summarization platform 100, such as the summary coordination logic 160 as described below.

More specifically, as shown in FIG. 1, the non-transitory storage medium 150 is adapted to store logic and data accessible to the processor(s) 145. The logic may include, but is not limited or restricted to (i) the summary coordination logic 160, (ii) graphical user interface (GUI) generation logic 170 configured to generate an interactive screen display (e.g., GUI) for rendering one or more summaries produced by the generative AI logic 132, and/or (iii) audio capture logic 180. The summary coordination logic 160 further comprises transcript generation logic 162, prompt generation logic 164, summary modification logic 166, and optionally anonymity control logic 168.

Additionally, the non-transitory storage medium 150 may be adapted with a local data store 190, which provides for storage of information such as (i) audio-to-text transcripts (e.g., transcript 136), (ii) summaries resulting from such transcripts (e.g., summary 138), (iii) prompts generated for transmission to the generative AI logic 132 (e.g., prompt 134), and/or (iv) a predefined group of keywords and/or set(s) of rules (e.g., salient content aggregation element 192). The salient content aggregation element 192 maintains a grouping of salient words and/or phrases, and optionally, a ruleset that, when processed by the summary modification logic 166, may be used to identify salient words and/or phrases to be annotated. According to one embodiment of the disclosure, the data store 190 may operate, at least in part, as a relational database or any other type of storage mechanism to supports correlation between the stored information.

The audio capture logic 180 is configured to directly capture audio content, for example, audio pertaining to a consultation between a health care professional (e.g., physician, nurse, assistant, etc.) and a patient. In some embodiments, however, the audio capture logic 180 may be configured to receive audio data from an external recording source or audio file input. The transcript generation logic 162 is configured to convert the audio data into a text-based transcript 136. Optionally, the anonymity control logic 168 is configured to recognize individually identifiable health information (e.g., patient name, patient address, etc.) within the transcript 136 and optionally redact or encrypt such information. In certain embodiments, the prompt generation logic 164 is designed to create a natural language processing (NLP) prompt that is transmitted to the content summarization service 130. The summary modification logic 166 is configured to recognize keywords 135 (e.g., medical terminology, dosage, numbers, drug names, frequency of treatment, etc.) from the summary 138 and annotate the keywords 135 by at least modifying their appearance (e.g., highlighting, adjusting a font style (bold, underline, etc.), changing font color, etc.) or appending information to give such keywords 135 prominence during a visual review of the summary 138.

Referring still to FIG. 1, an exemplary embodiment of the operational workflow of the AI-based summarization platform 100 is also shown. Herein, the prompt generation logic 164 is configured to generate the prompt 134 to be provided to a destination such as the content summarization service 130. The prompt 134 includes a set of instructions and/or contextual data provided to the generative AI logic 132 (e.g., LLM 132₁, LLM 132₂, etc.), which causes the LLM(s) 132₁-132₂ to perform one or more tasks. For this example, the task(s) may constitute a summary generation process that is adapted to generate the summary 138 from contextual data included as part of the prompt 134, such as content associated with the voice-to-text transcript 136 (e.g., portions of the transcript 136 or the entire transcript 136) and/or the summary format preferences 137 (e.g., Subjective, Objective, Assessment, and Plan (SOAP) notes, progress notes, medication list forms, etc.). For example, responsive to the transcript generation logic 162 converting a recorded voice dialogue into the transcript 136, the GUI generation logic 170 produces a display, visible on a display screen of the computing device 110, which allows for selection of (i) the transcript 136 (and/or any additional transcript) for summarization and (ii) a desired form for the summary 138 (i.e., summary format preferences 137). Upon selection of the transcript 136 and the summary format preference 137 for example, the prompt generation logic 164 of the summary coordination logic 160 produces the prompt 134, which may consist of the transcript 136 and the summary format preferences 137. Prompt 134 is provided to the content summarization service 130.

As further shown in FIG. 1, the summary coordination logic 160 receives the summary 138 based on the prompt 134 via the interface 140 and locally stores content from the summary 138 into the data store 190 for access by the summary modification logic 166. In the background, the summary modification logic 166 processes the information within the summary 138 by (i) identifying specific keywords 135 and/or phrases in the summary 138 that are maintained as part of the salient content aggregation element 192 or located in accordance with a stored ruleset and (ii) performing annotations on the identified salient keywords 135 and/or phrases. In some embodiments, the annotations are configured to enhance the visibility of the keywords 135 within the summary 138. In yet other embodiments, the summary modification logic 166 is configured to apply linking annotations to specific keywords and/or phrases that are catalogued within the salient content aggregation element 192. For example, medical conditions and guidelines categorized as salient keywords can include a hyperlink to an external webpage offering physicians rapid access to further details not included in the summary 138. Thereafter, the summary modification logic 166 is configured to locally store an annotated version of the summary 138 for access by the GUI generation logic 170 and/or store the annotated version of the summary into remote storage as part of an electronic health record.

The GUI generation logic 170 is configured to generate a GUI that provides a framework to display on a display screen annotated version of the summary 138 for analysis by the user. The user can view the modified summary content which includes the annotated keywords. In some embodiments, the annotated summary content will be displayed on a generated GUI with keywords 135 highlighted, bolded, or underlined throughout the text. The user can then manually review the annotated keywords for accuracy and revise the summary where necessary.

### III. Summary Coordination Logic Operability

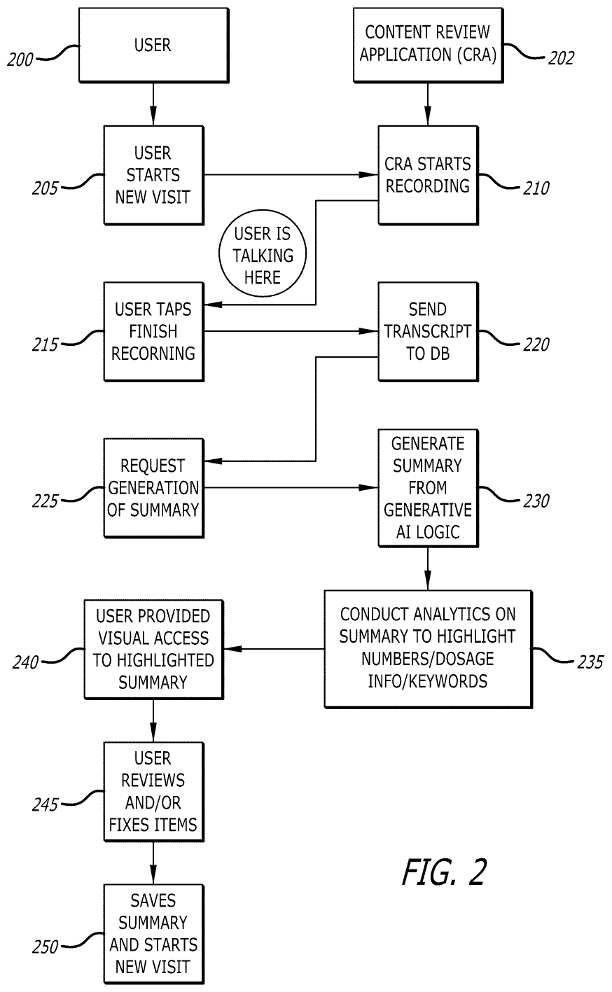

Referring now to FIG. 2, an exemplary flowchart operability of the summary coordination logic 160 of FIG. 1 is shown. Herein, the exchange of content between the user 200 and the summary coordination logic 160 operating as a software tool (also referred for this illustration as the content review application (CRA) 202) commences with a new visit by user, which results in a new session for recordation (blocks 205 and 210). The recordation of the communication exchange occurs, and upon completion, the user conducts an action to signal that the recorded session has been completed (block 215). Thereafter, the audio recordation is converted into a transcript for storage, where the conversion is accomplished by passing audio through an LLM that performs the translation automatically, upon which the recorded audio is deleted (block 220).

The generation of a summary of the transcript may be performed automatically or in response to a request by the user as shown (block 225). Upon commencing generation of the summary, the content review application (CRA) 202 (summary coordination logic 160) transmits the content of the transcript 136 to the content summarization service 130 via an application programming interface (API) established for that service (block 230). In an alternative embodiment, the content summarization service 130 is hosted locally on the CRA 202. In response to receiving the summary 138, the CRA 202 (summary coordination logic 160) parses the summary 138 to annotate salient content within the summary such as numbers, dosage, frequency, medicinal names, or other information directed to the particulars associated with the treatment prescribed by the health care professional (block 235). The selection of types of salient content may be hosted within a regular expression (regex) database that may be modified based on user feedback to add or remove identified salient content to be highlighted. The user is provided access to the annotated summary, where the user is permitted to alter its contents and save the resultant summary as part of the patient's electronic health record for later retrieval (blocks 240, 245 and 250).

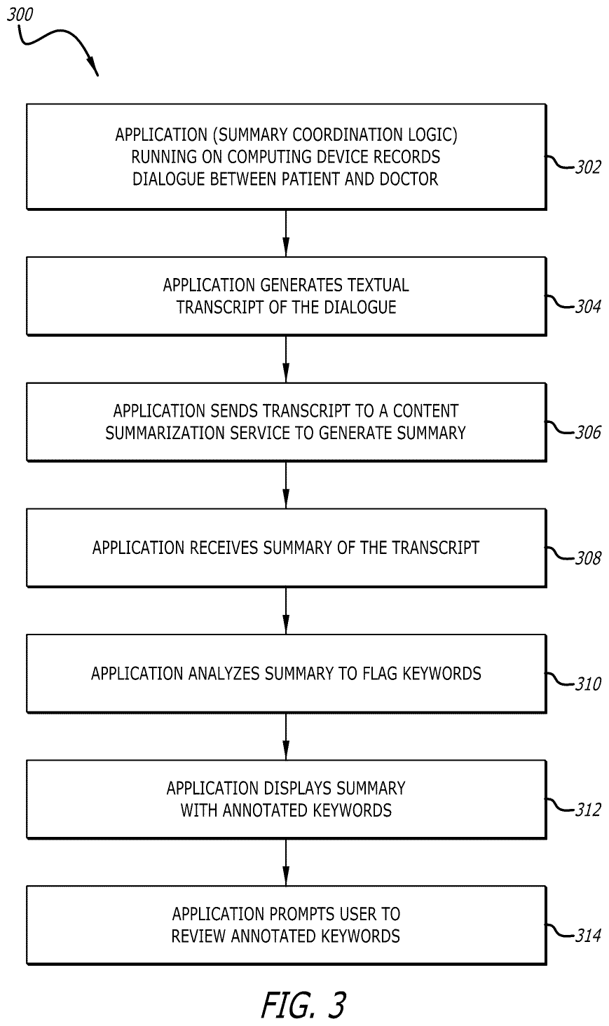

Referring to FIG. 3, an illustrative flow diagram of an exemplary process 300 conducted by an application, namely the summary coordination logic 160 of FIGS. 1-2, to facilitate summary generation and perform annotation (highlighting) operations on salient content within the returned summary is shown. Herein, this exemplary process 300 is adapted to generate an accurate, written summary of recorded verbal doctor-patient consultations with salient content such as certain medical terminology is annotated. The process 300 may be conducted, for example, by the computing device 110 of FIG. 1.

Each block illustrated in FIG. 3 represents an operation of the process 300. It should be understood that not every operation illustrated in FIG. 3 is required. In fact, certain operations may be optional to complete the process 300. The process 300 begins when a user starts a new visit initiating an application on a computing device, in some embodiments, to begin recording a doctor-patient consultation (block 302). In certain embodiments, the application running on a computing device is configured to record audio dialogue. In an alternative embodiment, the application can be configured to receive audio data from an external source (not shown). Once the user is finished recording, the application generates a transcript of the dialogue (block 304). In certain embodiments, the transcript generation logic 162 of FIG. 1 may implement a voice recognition algorithm to distinguish between voices of the patient and doctor. It should be noted that the generated transcript can also be configured to match the language of the received audio dialogue.

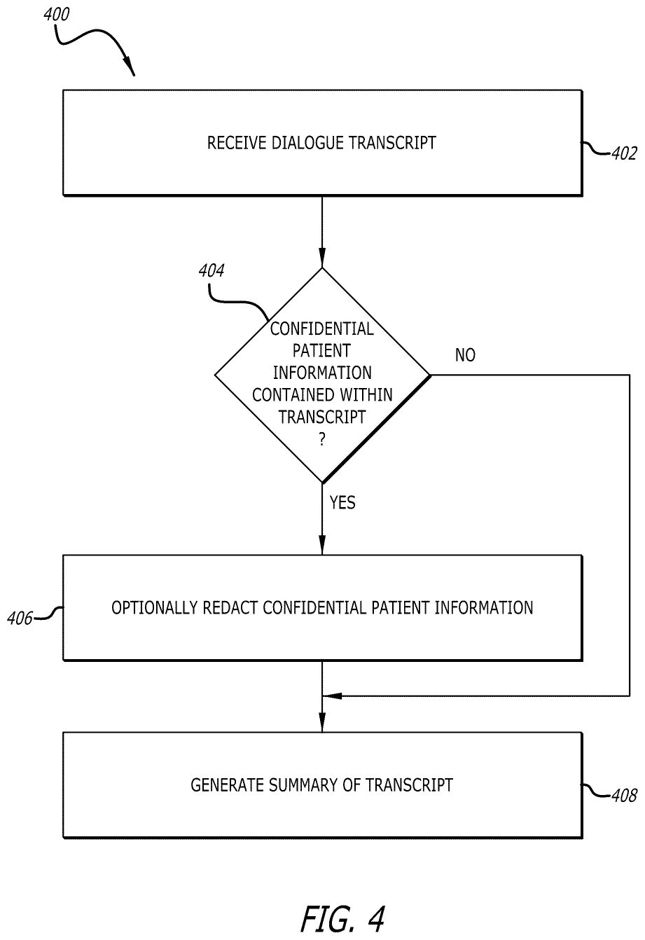

In certain embodiments, the anonymity control logic 168 of FIG. 1 may be configured to parse the transcript and redact or encrypt any sensitive or confidential patient information (see FIG. 4). For example, the anonymity control logic 168 of FIG. 1 can be configured to automatically detect and obscure individually identifiable health information such as names, addresses, etc. using predefined criteria and patterns. Additionally, this logic may employ encryption standards to secure any confidential data, such that only authorized users can access the full contents of the transcript.

The process continues with the operation of the application sending the transcript of the dialogue to a data store for temporary storage and/or sending the transcript to a content summarization service for process (block 306). In certain embodiments, the data store may be configured to retain the recording transcript for a specified period of time (e.g., sixty days). In other embodiments, a user may later access the transcript from the data store if, for example, the transcript summary generated by the content summarization service is incomplete or inaccurate. Automatically, or upon being prompted by a user, the application on the computing device transmits the transcript to the content summarization service and receives a summary of the transcript (block 308). Further details as to the summary generation process are discussed further with respect to at least FIG. 1.

The method continues with the operation of the application analyzing the summary to identify (flag) specific keywords or phrases that denote salient content within the summary (block 310). For example, the application may be configured to assess whether any keywords and/or phrases maintained within a salient content aggregation element maintained within the data store are present in the generated summary. If the application identifies a word or phrase within the summary as a keyword, the application will flag that keyword. For example, the application can be configured to flag numbers, dosage information, prescriptions, patient treatment plans, etc., if those parameters appear within the summary. In some embodiments, the flagging process includes altering a visual representation of the keywords and/or phrases (salient content) within the summary for display to the user via a GUI (block 312).

The application then prompts the user to review highlighted keywords and make revisions (block 314). The application then saves the summary in a data store and starts a new visit (not shown). It is contemplated that the application could be configured to require that all highlighted keywords may be reviewed manually or through an automated process, such as DocuSign application process.

Referring now to FIG. 4, a flow diagram illustrating the optional process employed by the data anonymity logic is shown in accordance with some embodiments. The operations set forth in blocks 402-408 are optional and designed to obfuscate confidential patient information in the transcript being sent to the content summarization service, but to re-insert the confidential patent information back into the summary for the anonymized data.

In certain embodiments, the anonymity control logic 168 is configured to identify confidential information and substitute it with a random unique identifier. It may recognize patient names based on machine learning analysis of historical data, or it could identify sensitive data through predefined privacy filters and pattern recognition techniques. Once recognized, the anonymity control logic stores the correlation between the unique identifier and the specific keyword or phrase in data store 190. After the summary coordination logic 160 receives the summary 138 from the content summarization service 130, the anonymity control logic 168 may be configured to access the data store 190 to decode the text by reinserting the previously stored confidential information removed from the transcript 136 provided in the prompt 134.

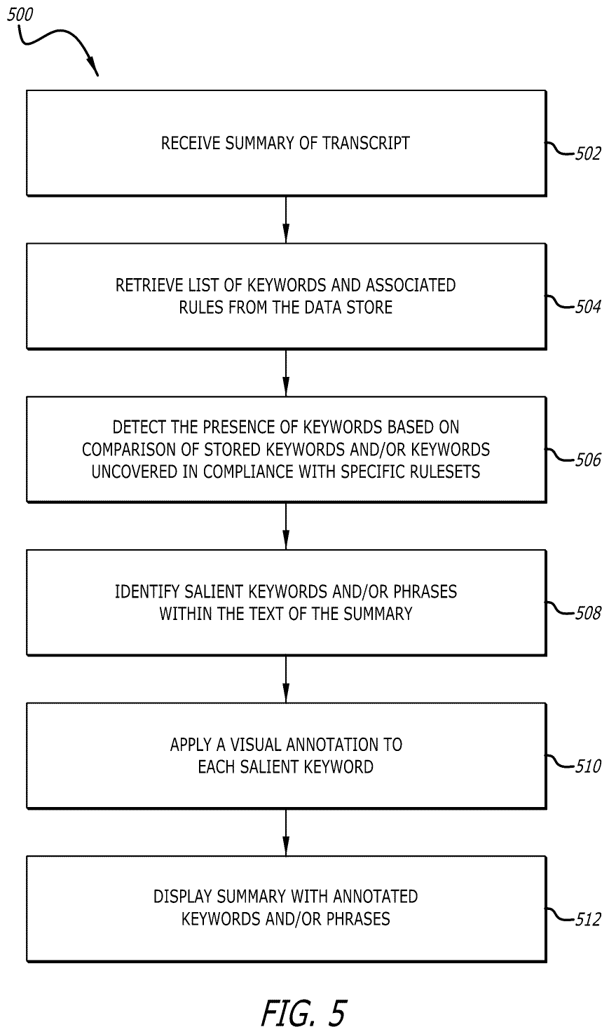

Referring now to FIG. 5, a flow diagram illustrating the keyword flagging and highlighting process implemented by the generative AI summarization platform of FIGS. 1-2 is shown according to some examples. FIG. 5 illustrates an example process 500 for receiving a summary of a transcript, and flagging the transcript for any keywords. The example process 500 may be implemented, for example, by a computing device that comprises one or more processors and non-transitory computer-readable medium. The non-transitory computer readable medium may store instructions that, when executed by the processor(s), cause the processor(s) to perform the operations of the illustrated process 500.

Each block illustrated in FIG. 5 represents an operation of the process 500. It should be understood that not every operation illustrated in FIG. 5 is required. In fact, certain operations may be optional to complete aspects of the process 500. The process 500 begins when the application receives a summary 138 of the transcript from a content summarization service 130 of FIG. 1 (block 502). In certain embodiments, the application (summary modification logic 166 of the summary coordination logic 160)) has access to specific keywords and/or phrases maintained as part of the salient content aggregation element 192 or located in accordance with a stored ruleset (block 504). In other embodiments, the data storage of keywords may be customized by a specific user. In yet other embodiments, keywords are determined by an ML model trained on historical data and the salient content aggregation element 192 being updated by the ML model.

After one or more salient keywords and/or phrases are detected, the application applies visual highlights to each of these salient keywords and/or phrases to produce a revised summary with the annotated salient content (blocks 506, 508, 510). The revised summary is subsequently displayed to the user for review, modification as needed, and storage as part of the electronic health record (block 512).

### IV. Embodiments of the Computing Device/Summary Generation

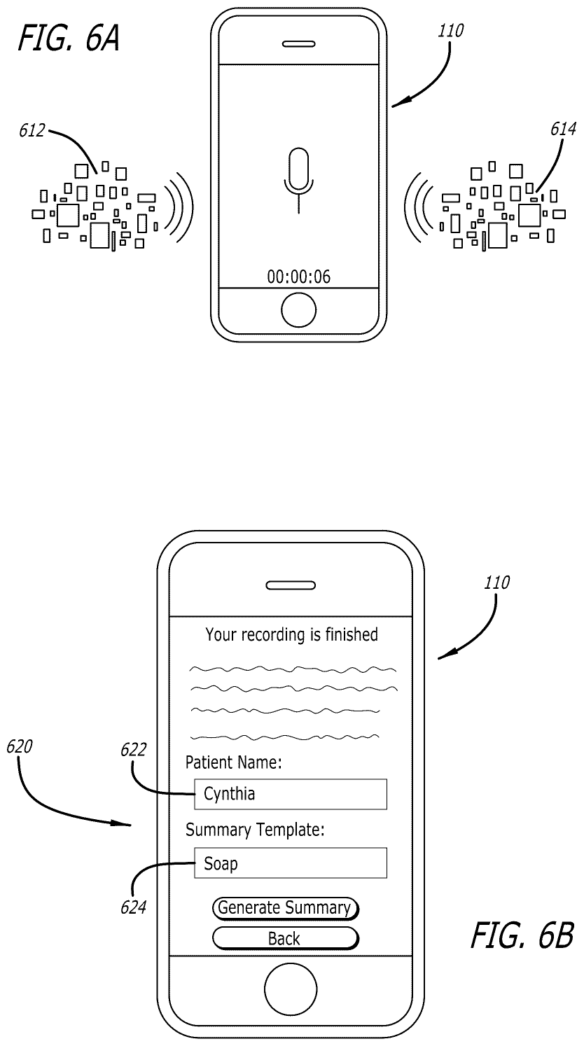

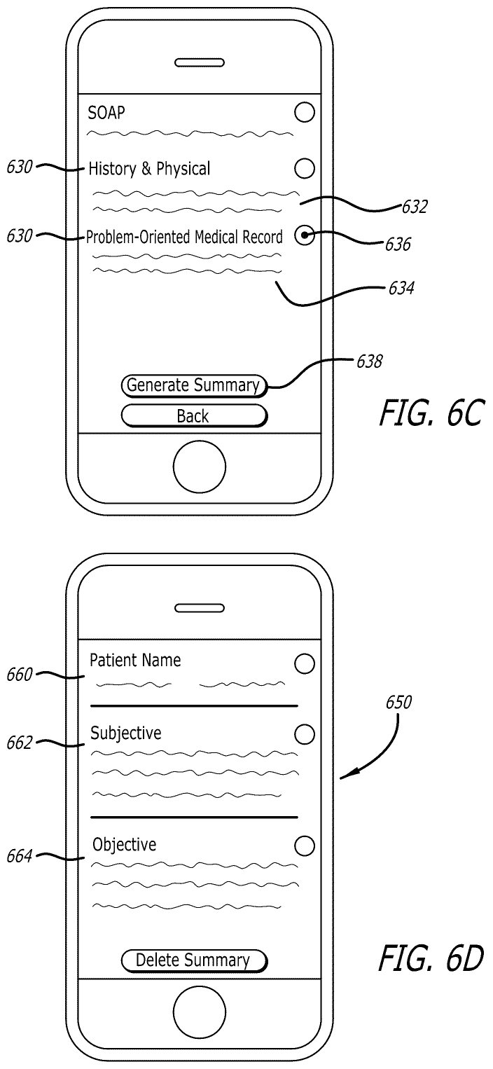

Referring to FIG. 6A, an exemplary perspective view of an illustrative embodiment of the computing device 110 adapted with summary coordination logic 160 of FIG. 1 is shown. Herein, the computing device 110 (represented as a smartphone) operates as a Health Insurance Portability and Accountability (HIPAA) compliant tool that transforms content of communication sessions 612, such as a doctor-patient session, into an electronic health record (HER) ready summary 614, where the doctor can focus entirely on the patient while the summary coordination logic 160 deployed within the computing device 110 captures audio content from the session.

As shown in FIG. 6B, once the session is over, the GUI generation logic 170 of FIG. 1 is configured to generate a visualization 620, which includes a first display object 622 for entry of the patient's name and a second display object 624 for selection of a summary template (Subjective, Objective, Assessment, Plan-SOAP). As shown in FIG. 6C, the summary templates 630 may be predetermined or customized by the user. In particular, for this embodiment, a first selectable summary template 632 may be configured to generate a template associated with an EHR pertaining to the patient history while a second selectable summary template 634 may be directed to a separate record directed to a specific problem that caused the patient to visit the doctor. The user (doctor, physician assistant, administrator, etc.) may select one of these summary templates 630 by selection of a corresponding selection display element (e.g., element 636), and upon selection of the ‘generate summary’ display object 638, a comprehensive summary (e.g., EHR) in a chosen summary template format is created.

Referring to FIG. 6D, an exemplary embodiment of a visualization 650 of a selected summary template (e.g., customized template 634 of FIG. 6C), which is populated with the transcript data, is shown. The transcript data is categorized into different sections 660, 662 and 664, where these sections 660, 662 and 664 may be modifiable by the user on the computing device 110 or another computing device (e.g., laptop in communication with the computing device 110 communicatively coupled thereto). The summary coordination logic 160 may be further configured to annotate certain terms (dosage, medicine name, etc.) and generate a pop-up to identify a prescription mistake to help avoid wrong dosages or incorrect instructions.

In the foregoing description, the invention is described with reference to specific exemplary embodiments thereof. It will, however, be evident that various modifications and changes may be made thereto without departing from the broader spirit and scope of the invention as described herein.

What is claimed is:

1. A computing device, comprising:
one or more processors; and
a non-transitory storage medium communicatively coupled to the one or more processors, the non-transitory storage medium comprises summary coordination logic including (i) prompt generation logic configured to generate a natural language processing (NLP) prompt, based on a text-based transcript converted from audio data, for transmission to a content summarization service and (ii) summary modification logic configured to recognize one or more keywords within a summary provided from the content summarization service and annotate the one or more keywords by at least modifying an appearance of the one or more keywords or appending information to give the one or more keywords prominence during a visual review of the summary.

2. The computing device of claim 1, wherein the one or more keywords recognized by the summary modification logic are directed to medical terminology, dosage, numbers, drug names, or frequency of treatment.

3. The computing device of claim 1, wherein the summary modification logic is configured to modify the appearance of the one or more keywords by at least highlighting the one or more keywords, adjusting a font style of the one or more keywords, or changing a color of a font associated with the one or more keywords.

4. The computing device of claim 1, wherein the summary coordination logic of the non-transitory storage medium further comprises transcript generation logic configured to convert the audio data into the text-based transcript.

5. The computing device of claim 1, wherein the summary coordination logic of the non-transitory storage medium further comprises anonymity control logic configured to recognize individually identifiable health information within the text-based transcript and redact or encrypt the individually identifiable health information prior to generating the NLP prompt.

6. The computing device of claim 5, wherein the individually identifiable health information includes a patient name, a patient address, a birth date, an address, or social security number.

7. A non-transitory storage medium including software that, upon execution by one or more processors, generates a summary originating from a recordation of audio data, the non-transitory storage medium comprising:
prompt generation logic configured to generate a natural language processing (NLP) prompt, based on a text-based transcript converted from the audio data, for transmission to a content summarization service;
summary modification logic configured to recognize one or more keywords within a summary provided from the content summarization service and annotate the one or more keywords by at least modifying an appearance of the one or more keywords or appending information to give the one or more keywords prominence during a visual review of the summary.

8. The non-transitory storage medium of claim 7, wherein the one or more keywords recognized by the summary modification logic are directed to medical terminology, dosage, numbers, drug names, or frequency of treatment.

9. The non-transitory storage medium of claim 7, wherein the summary modification logic is configured to modify the appearance of the one or more keywords by at least highlighting the one or more keywords, adjusting a font style of the one or more keywords, or changing a color of a font associated with the one or more keywords.

10. The non-transitory storage medium of claim 7 further comprising transcript generation logic configured to convert the audio data into the text-based transcript.

11. The non-transitory storage medium of claim 7 further comprising anonymity control logic configured to recognize individually identifiable health information within the text-based transcript and redact or encrypt the individually identifiable health information prior to generating the NLP prompt.

12. The non-transitory storage medium of claim 11, wherein the individually identifiable health information includes a patient name, a patient address, a birth date, an address, or social security number.

13. A computerized method, comprising:
generate a natural language processing (NLP) prompt based on a text-based transcript converted from audio data;
transmitting the NLP prompt to a content summarization service that generates a summary based on the NLP prompt; and
recognizing one or more keywords within the summary and annotating the one or more keywords by at least modifying an appearance of the one or more keywords or appending information to give the one or more keywords prominence during a visual review of the summary.

14. The computerized method of claim 13, wherein the one or more keywords are directed to medical terminology including dosage, drug names, and frequency of treatment.

15. The computerized method of claim 13, wherein the modifying of the appearance of the one or more keywords includes at least highlighting the one or more keywords.

16. The computerized method of claim 13, wherein the modifying of the appearance of the one or more keywords includes adjusting a font style of the one or more keywords.

17. The computerized method of claim 13, wherein the modifying of the appearance of the one or more keywords includes changing a color of a font associated with the one or more keywords.

18. The computerized method of claim 13, wherein prior to generating the NLP prompt, the computerized method further comprises converting the audio data into the text-based transcript.

19. The computerized method of claim 13 further comprising:
prior to generating the NLP prompt, recognizing individually identifiable health information within the text-based transcript and redact or encrypt the individually identifiable health information.

20. The computerized method of claim 19, wherein the individually identifiable health information includes a patient name, a patient address, a birth date, an address, or social security number.
